(19) United States
(12) Patent Application Publication
(10) Pub. No.: US 2004/0091553 A1
(43) Pub. Date: May 13, 2004

(54) Composition and method for mammary disinfection during winter conditions

(75) Inventor: Chris Foret, Shawnee Mission, KS (US)

Correspondence Address: HOVEY WILLIAMS LLP, 2405 GRAND BLVD., SUITE 400, KANSAS CITY, MO, 64108, US

(73) Assignee: WEST AGRO, INC

(21) Appl. No.: 10/293,410

(22) Filed: Nov. 12, 2002

(51) Int. Cl. A61K 33/36

(52) U.S. Cl. 424/667

(57) ABSTRACT

Improved aqueous, ready to use iodine teat dip compositions are provided which are specially formulated for winter time use to permit application to cows teats without freezing while at the same time affording adequate germicidal activity. The compositions include from about 0.01-2% by weight iodine, from about 0.01-3% by weight iodide ion, and from about 35-75% by weight of an additive selected from the group consisting of glycerin and a mixture of at least about 10% by weight glycerin based upon the total weight of the composition and one or more C2-C7 polyhydric alcohols different than glycerin and each containing at least 2 hydroxyl groups, said composition containing no more than about 45% by weight propylene glycol when propylene glycol is one of said polyhydric alcohols. The compositions preferably have free iodine values of from about 1-60 ppm at 25° C. and from about 0.5-20 ppm at 4° C.

### BACKGROUND OF THE INVENTION

1. Field of the Invention

The present inventions is broadly concerned with ready to use teat dip compositions especially designed for low temperature winter time use. More particularly, the invention is concerned with such use compositions, and methods of employing the same, wherein the compositions include respective quantities of iodine, iodide ion and a relatively large amount of an additive for lowering the freezing point of the composition while maintaining adequate free iodine values even at low temperatures. Such an additive may be glycerin or a mixture of glycerin and one or more C2-C7 polyhydric alcohols.

2. Description of the Prior Art

Mastitis is a major problem in the dairy industry, and can be caused by contagious or environmental organisms. While mastitis can be cured in most cases by antibiotics, the preferred approach for mastitis control is by way of prevention. Mastitis prevention requires clean, dry milking facilities and careful disinfection of related equipment. Also, germicidal teat dips are used before and sometimes after milking as a disinfection technique. These teat dips are often aqueous compositions containing iodine as the sole or principal disinfecting agent.

Winter conditions present a number of formidable problems for mastitis control. Wet teats coated with aqueous dip are especially susceptible to frostbite, and indeed prior dips containing large amounts of water can actually freeze on the teats when applied. This problem has been so severe that dairymen often simply cease dipping teats during low temperature conditions. While this avoids the freezing problem, it does tend to increase the rate and severity of mastitis infections.

Special dips have been formulated for winter time use which employ significant amounts of organic solvents, which often have a lower rate of heat transmission than water. Propylene glycol has been used in this context, see, PCT publication WO 02/28,180 which employs propylene glycol and a fatty acid germicide. PCT publication WO 01/82,702 describes compositions having very high concentrations of propylene glycol with iodine. Such compositions exhibit low freezing points but tend to adversely affect teat skin during winter conditions.

U.S. Pat. No. 6,152,229 describes stable glycerin iodine dilutable concentrates containing 30-87% by weight glycerin. However, these compositions are not ready to use products and are not designed for winter time use.

### SUMMARY OF THE INVENTION

The present invention overcomes the problems outlined above and provides improved ready to use teat dip compositions that are particularly adapted for winter time use, having very low freezing points while at the same time maintaining adequate free iodine levels for enhanced germicidal activity. Broadly speaking, the compositions of the invention include water, from about 0.01-2% by weight iodine and from about 0.01-3% by weight iodide ion. The compositions also include an additive serving to decrease freezing points and maintain free iodine levels and which is selected from the group consisting of glycerin and mixtures of at least about 10% by weight glycerin based upon the total weight of the composition and one or more C2-C7 polyhydric alcohols different than glycerin and each containing at least 2 hydroxyl groups, said composition containing no more than about 45% by weight propylene glycol when propylene glycol is one of said polyhydric alcohols. Such compositions can be used as teat dips which may be applied by any suitable technique (e.g. dipping, spraying or foaming) to cow's teats for mastitis prevention, and are especially suited for such application during periods where the ambient temperature is below about 5° C. or even below about −15° C. The compositions of the invention normally have a free iodine content of from about 1-60 ppm at a temperature of 25° C. and a content from about 0.5-20 ppm at 4° C.

In preferred forms, the use compositions have an iodine content of about 0.2-1.1% by weight, an iodide ion content from about 0.5-1.5% by weight, with the additive being present at a level from about 35-75% by weight. The iodide ion source is preferably an alkali metal iodide such as sodium iodide, potassium iodide and hydriodic acid. As indicated above, the additive may consist essentially of glycerin. Alternately, the additive may be made up of a mixture of glycerin and one or more C2-C7 polyhydric alcohols or polyethylene glycol. This includes the alkylene glycols (e.g., ethylene and propylene glycol, diethylene glycol, and dipropylene glycol) and polyols such as C2-C7 polyhydric alcohols (e.g., the glycerols having 3 hydroxyl groups) and the so called sugar alcohols having the general formula CH₂OH(CHOH)ₙCH₂OH, where n may range from 2-5 (e.g., sorbitol).

As used herein, “free iodine” is the concentration of 12 which is not complexed with other species such as iodide ion I₃⁻. A certain concentration of free iodine is always present in iodine solutions because of equilibrium reactions such as

I₃ [symbol] I₂+I⁻

or in general, I₂ (complexing agent) [symbol] I₂+complexing agent. Free iodine is preferably determined by the method of Winicov et al., Proc. Int. Symposium on Providone, University of Kentucky College of Pharmacy, pp. 186-92 (1983), incorporated by reference herein.

It is also advantageous that the compositions of the invention remain stable over extended periods of time, preferably at least about 3 months and more preferably at least about 6 months. As used herein “stability” refers to the ability of the compositions to remain as substantially single phased, homogeneous solutions throughout a given storage period at room temperature while retaining at least about 80% of the starting iodine concentrations thereof.

### BRIEF DESCRIPTION OF THE DRAWINGS

FIG. 1 is a comparative graph illustrating average skin condition scores obtained when using a composition in accordance with the invention, versus a commercially available winter teat dip product;

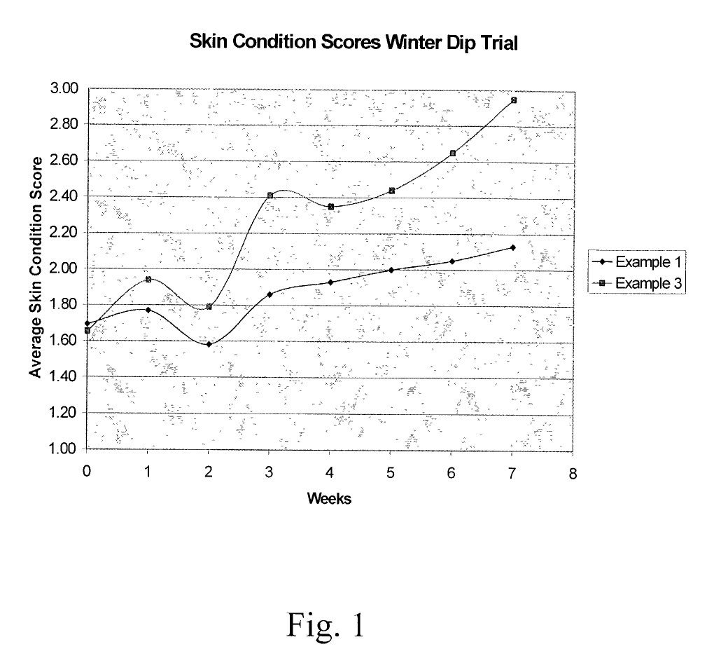

FIG. 2 is a comparative graph illustrating average teat end scores obtained when using a composition in accordance with the invention, versus a commercially available winter teat dip product; and

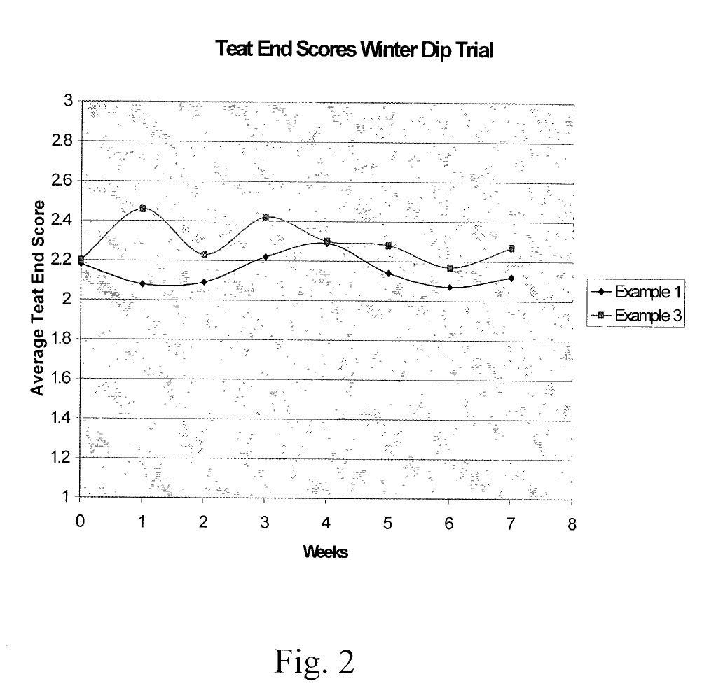

FIG. 3 is a comparative graph illustrating average teat orifice scores obtained when using a composition in accordance with the invention, versus a commercially available winter teat dip product.

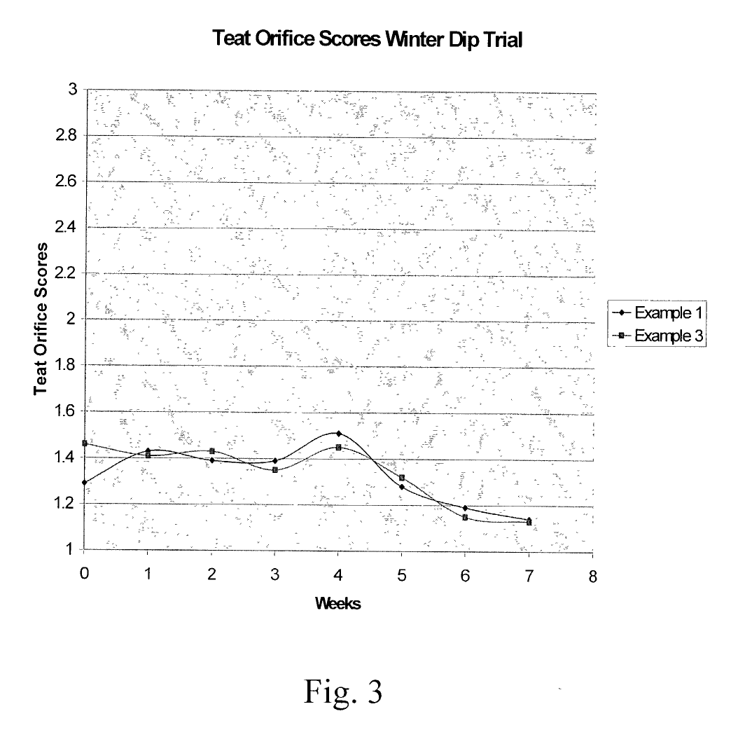

### DETAILED DESCRIPTION OF THE PREFERRED EMBODIMENT

The following examples set forth preferred teat dip compositions in accordance with the invention, as well as test results demonstrating the efficacy thereof. It is to be understood, however, that these examples are provided by way of illustration and nothing therein should be taken as a limitation upon the overall scope of the invention.

### EXAMPLE 1

A teat dip composition was prepared by mixing together 50% by weight glycerin, 1% by weight iodine, 1.35% by weight sodium iodide, 0.1% by weight dioctyl sodium sulfosuccinate, 0.5% by weight decyl polyglucose and sufficient water to bring to a total of 100% by weight. The free iodine content of this composition at 25° C. was 17 ppm and at 4° C. was 3.6 ppm after 1 week at 25° C. The freezing point of this composition is −32.3° C.

### EXAMPLE 2

Another teat dip composition was prepared as set forth in Example 1 and included 45% by weight glycerin, 1% by weight iodine, 1.35% by weight sodium iodide, 0.1% by weight dioctyl sodium sulfosuccinate, 0.5% by weight decyl polyglucose and sufficient water to bring to a total of 100% by weight. The free iodine content of this composition at 25° C. was 21.7 ppm and at 4° C. was 5.7 ppm after 1 week at 25° C. The freezing point of this composition is −26.5° C.

### EXAMPLE 3

A commercially available teat dip product (Derma Kote lot 0131104R) containing about 75% by weight propylene glycol and about 0.5% by weight iodine and other ingredients was analyzed for free iodine content. The commercial preparation had a free iodine content of 0.16 ppm at 25° C. and less than 0.05 ppm free iodine at 4° C. The freezing point of this composition is less than −70° C.

### EXAMPLE 4

A skin condition trial was performed on dairy cattle to compare the overall effects of the Example 1 formula in accordance with the invention versus the Example 3 commercial preparation. The trial was performed under winter conditions over a 7-week period. The cows remained outside in the cold when not being milked in the parlor; at night the cows were bedded in a barn. A temperature recorder at the trial site established that the ambient temperature was below freezing (32° F.) for approximately 20% of the time during 7-week trial period.

The teat skin condition, teat end condition and degree of teat hyperkeratosis was evaluated by a trained observer having no knowledge of the identity of the test products. The scoring was based on a relative scale using the parameters set forth below:

Teat Skin Conditioning Scores

| | |
|---|---|
| 1 | Smooth supple skin free from scales cracks or chapping. |
| 2 | Slightly drying of skin with a superficial flaking. |
| 3 | More sever drying, with early cracks present. Skin cracks do no |
| | have severe red fissures at the base. |
| 4 | More teat skin is chapped with pronounced cracks present. |
| | Redness, indicating inflammation is present. Some scabbed; healing |
| | lesions may be present |
| 5 | Sever skin damages with deep chaps and open ulcerative lesions or |
| | scabs. |

Teat End Conditioning Scores
Rough teat end:

| | |
|---|---|
| 1 | Smooth teat end and sphincter with no evidence of irritation. |
| 2 | Slightly drying of teat end. |
| 3 | Teat end sphincter is roughened with slight cracks, but no redness |
| | is present. |
| 4 | Teat end skin is chapped and cracked. Redness, indicating |
| | inflammation is present. Some scabbed; healing lesions may be |
| | present. |
| 5 | Teat end is severely damaged and ulcerative with scabs or open |
| | lesions. |

Smooth teat end - Hyperkeratosis Teat Orifice:

| | |
|---|---|
| 1 | No Ring. |
| 2 | Slight raised ring. |
| 3 | Moderate raised ring. |
| 4 | Thick raised ring. |

Exhibits 1, 2 and 3 graphically depict the average score for the skin conditions, teat end condition and teat hyperkeratosis analyses of this Example.

The data derived from these experiments were compared using the Mann-Whitney U test for ordinal data. The average score for each cow was calculated over the 7-week period minus the initial at week 0. This accounts for any slight difference between the groups of cows at the beginning of the trial.

The statistical analyses of the skin condition data resulted in a p value of 0.0000. This indicates a probability >99.99% that there is a significant difference in the test skin data for the 50% glycerin dip Example 1 and the 75% propylene glycol dip Example 3. The statistical analyses of the teat end condition data resulted in a p value of 0.14. This indicates that the Example 1 dip gave better teat end results compared to the Example 3 dip, with a probability of 86% that the Example 1 dip is significantly different than the Example 3 dip. No significant difference was observed for the teat orifice scores between the Example 1 and Example 3 dips.

### EXAMPLE 5

A series of sample teat dip compositions (Samples 4-23) were prepared containing the ingredients set forth in Tables 1-3, wherein each such sample also included 1.00% iodine and 2.35% by weight sodium iodide (57%), and q.s. water to give a total of 100% by weight. The resulting iodine concentration is 1% for all examples in Tables 1-3.

The Samples were stored for 1 week at ambient temperature (about 25° C.), whereupon the samples were tested for free iodine content at 25° C. Thereafter, the samples were refrigerated to a temperature of 4° C. and again tested for free iodine content. These results are set forth in Table 1.

Additional quantities of the Samples 4-23 were stored at an elevated temperature of 50° C. for 1 week in order to obtain accelerated storage stability data. At the end of the 1 week storage, the samples were cooled to 25° C. and the free iodine thereof was determined. Next, these stored samples were further cooled to 4° C. and free iodine values were again determined. This data is recorded in Table 2.

The freezing points of the respective Samples 4-23 were also measured and these data are given in Table 3.

TABLE 1
1 Week at 25° C. Samples

| | % Propylene | % | ppm Free Iodine | ppm Free Iodine |
|---|---|---|---|---|
| Sample | Glycol | Glycerin | at 25° C. | at 4° C. |
| 4 | 0 | 0 | 203.4 | 110.1 |
| 5 | 25 | 0 | 53.8 | 25.7 |
| 6 | 30 | 0 | 35.0 | 11.8 |
| 7 | 35 | 0 | 18.8 | 6.1 |
| 8 | 40 | 0 | 12.3 | 4.3 |
| 9 | 45 | 0 | 6.2 | 3.0 |
| 10 | 50 | 0 | 4.6 | 2.6 |
| 11 | 60 | 0 | 2.0 | 1.3 |
| 12 | 0 | 35 | 56.9 | 17.7 |
| 13 | 0 | 40 | 47.8 | 13.8 |
| 14 | 0 | 45 | 33.8 | 10.1 |
| 15 | 0 | 50 | 32.5 | 6.6 |
| 16 | 0 | 55 | 21.6 | 5.6 |
| 17 | 0 | 60 | 17.4 | 4.3 |
| 18 | 0 | 65 | 13.4 | 2.6 |
| 19 | 0 | 70 | 6.8 | 2.4 |
| 20 | 0 | 75 | 4.5 | 1.7 |
| 21 | 30 | 10 | 18.2 | 6.3 |
| 22 | 20 | 20 | 28.5 | 5.3 |
| 23 | 10 | 30 | 37.7 | 9.0 |

TABLE 2
1 Week at 50° C. Samples

| | % Propylene | % | ppm Free Iodine | ppm Free Iodine |
|---|---|---|---|---|
| Sample | Glycol | Glycerin | at 25° C. | at 4° C. |
| 4 | 0 | 0 | 202.1 | 94 |
| 5 | 25 | 0 | 50.4 | 24.2 |
| 6 | 30 | 0 | 33.3 | 11.3 |
| 7 | 35 | 0 | 5.19 | 2.6 |
| 8 | 40 | 0 | 3.6 | 1.8 |
| 9 | 45 | 0 | 2.1 | 1.5 |
| 10 | 50 | 0 | 1.8 | 1.2 |
| 11 | 60 | 0 | 1.1 | 1.3 |
| 12 | 0 | 35 | 54.0 | 15.7 |
| 13 | 0 | 40 | 44.0 | 16.3 |
| 14 | 0 | 45 | 31.7 | 9.3 |
| 15 | 0 | 50 | 24.7 | 6.6 |
| 16 | 0 | 55 | 20.4 | 4.2 |
| 17 | 0 | 60 | 16.7 | 3.3 |
| 18 | 0 | 65 | 12.6 | 2.6 |
| 19 | 0 | 70 | 6.2 | 2.5 |
| 20 | 0 | 75 | 4.9 | 1.9 |
| 21 | 30 | 10 | 15.6 | 2.8 |
| 22 | 20 | 20 | 27.5 | 10.0 |
| 23 | 10 | 30 | 24.8 | 8.5 |

TABLE 3
Freezing Point Data

| Sample | % Propylene Glycol | % Glycerin | Freezing Point ° C. |
|---|---|---|---|
| 4 | 0 | 0 | — |
| 5 | 25 | 0 | −11 |
| 6 | 30 | 0 | −18 |
| 7 | 35 | 0 | −21 |
| 8 | 40 | 0 | −25 |
| 9 | 45 | 0 | −33 |
| 10 | 50 | 0 | −37 |
| 11 | 60 | 0 | −69 |
| 12 | 0 | 35 | −15 |
| 13 | 0 | 40 | −19 |
| 14 | 0 | 45 | −23 |
| 15 | 0 | 50 | −29 |
| 16 | 0 | 55 | −33 |
| 17 | 0 | 60 | −59 |
| 18 | 0 | 65 | <−70 |
| 19 | 0 | 70 | <−70 |
| 20 | 0 | 75 | <−70 |
| 21 | 30 | 10 | −24 |
| 22 | 20 | 20 | −23 |
| 23 | 10 | 30 | −21 |

As reflected in the foregoing tables, the concentration of organic components in the ready to use teat dip compositions of the invention may be used to control the free iodine contents thereof. For germicidal efficacy during winter conditions, the free iodine value of the use compositions should preferably be at least 0.5 ppm at 4° C. The preferred maximum free iodine content depends upon the concentration of available iodine in the dip. In the case of the 1% iodine solutions of Tables 1-3, a maximum free iodine content of about 60 ppm is deemed appropriate. For 0.1% iodine solutions, a free iodine content of 100 ppm could be used without irritation to skin.

The free iodine data of Tables 1 and 2 confirms that by appropriate adjustment of the glycerin concentration between 35-75% by weight, the free iodine concentration can be kept in the range of from about 0.5-20 ppm at 4° C. Glycerin concentrations of above about 75% give use compositions with low free iodine concentrations which are not as effective as germicides. Solutions with glycerin concentrations below 35% yield free iodine concentrations above 60 ppm at 25° C., which can be irritating to the skin when using 1% iodine solutions. In contrast, propylene glycol concentrations above 45% by weight give free iodine values below 1.5 ppm at 4° C. Accordingly, for the current invention, the optimum range for propylene glycol or other polyhydric alcohol use is in the neighborhood of 25-45% by weight based upon the total weight of the composition, to give a reasonable free iodine value during the winter conditions (e.g. 4° C.) or normal ambient temperatures (e.g. 25° C.).

The free iodine of aqueous solutions containing 35-75% glycerin or other polyhydric alcohols is controlled mainly by the concentration of the polyhydric alcohol. However, the equilibrium free iodine concentration is also affected by the ratio of iodide to iodine. A higher ratio of iodide to iodine tends to lower the free iodine concentration. In addition, other ingredients such as surface-active agents will have some effect on the overall free iodine concentration. Compare the free iodine obtained for Examples 1 and 2 to the values of Samples 13 and 14 in Table 1. This demonstrates that the addition of surface active agents and buffering agents have some effect on the equilibrium free iodine concentration. Therefore, through a combination of polyhydric alcohol concentration, iodide, additives, and iodine concentration the free iodine can be controlled to between about 0.5-20 ppm at 4° C. and 1-60 ppm at 25° C.

As is apparent, free iodine values are a result of a chemical equilibrium and such values may vary with time and temperature. However, the free iodine values in use compositions containing propylene glycol may be somewhat unstable. For example, compare the free iodine values for the propylene glycol samples 7-10 in Table 1 where the Samples were aged for one week at 25° C. versus these same samples in Table 2 aged for 1 week at 50° C. This demonstrates that the free iodine values dropped to less than half after aging for 1 week at 50° C., in comparison to the 25° C. aged Samples. On the other hand, use compositions using glycerin alone or mixtures of glycerin and propylene glycol exhibit much more stable free iodine values. Compare the free iodine values for Samples 12-23 in Tables 1 and 2.

The concentration of glycerin and/or other polyhydric alcohol additive will also effect the freezing point of the use composition. For products intended for primary use in winter conditions it is beneficial if the product does not readily freeze. If the product does not freeze then it will continue to be free flowing and slowly drain off the skin leaving a thin layer of germicidal product. The freezing points for the various concentrations for glycerin and propylene glycol are listed in Table 3. Generally, higher concentrations of glycol will lower the freezing points most significantly. Accordingly, the optimum concentration of glycol to be used in a winter germicidal teat dip composition should be optimized according to the opposing factors of lowering the free iodine concentration on the one hand, or lowering the freezing point due to increasing glycol concentrations on the other hand. Freezing points of −15° C. or below prevent a teat dip from freezing during most winter conditions encountered in practice thus, a solution containing 35% glycerin would exhibit adequate freezing point depression while giving excellent free iodine values.

While the invention has been described in specific terms in connection with the foregoing Examples, it will be understood that the invention is not so limited. For example, compositions of the invention may include a variety of other ingredients such as thickening agents, surface active agents, buffering agents and colorants. Typical thickening agents can be selected from the group consisting of cellulose derivatives such as hydroxy ethylcellulose and carboxy methylcellulose, sodium alginate, xanthan gum and mixtures thereof. Exemplary surface active agents include sulfonates such as the alkyl sulfonated, aryl sulfonates, alkyl aryl sulfonates, alkyl diphenyloxide disulfonate, dialkyl sodium sulfosuccinates, sulfonated amphoterics such as alkylamphohydroxy propyl sulfonate, polysulfonates such as lignosulfate C8-C 16 alkyl polyglycosides, sodium alcohol sulfates, and mixtures thereof. Suitable buffering agents would include C2-C 10, fatty acids, di-and tri-carboxyl acids (e.g. citric acid) and inorganic acids (e.g. phosphoric acid).

Important ingredients and properties characterizing the ready to use compositions of the invention, with approximate broad and more preferred ranges are set forth in the following Table 4.

TABLE 4
Use Compositions

| Ingredient | Broad Range | | Preferred Range | |
|---|---|---|---|---|
| Iodine | 0.01-2.0% | by wt. | 0.2-1.1% | by wt. |
| Iodide ion | 0.01-3% | by wt. | 0.25-1.5% | by wt. |
| C2-C7 polyhydric alcohol or | 35-75% | by wt. | 40-65% | by wt. |
| polyethylene glycol¹ | | | | |
| Thickening agents | 0-0.3% | by wt. | 0-0.1% | by wt. |
| Surface Active Agents | 0-5% | by wt. | 0-2% | by wt. |
| Water | q.s. | | q.s. | |
| Freezing Point | −15° C. and below | | −19° C. and below | |
| Temperature of Use | Below +5° C. | | Below 0° C. | |
| Free Iodine Concentration | 0.5-20 | ppm | 3-12 | ppm |
| (4° C.)² | | | | |
| Free Iodine Concentration | 1-60 | ppm | 5-40 | ppm |
| (25° C.)² | | | | |

¹May be in the form of a single polyhydric alcohol (e.g., glycerin) or a mixture thereof.
²Measured after 1 week at 25° C. or 1 week at 50° C. for 1% iodine solutions.

I claim:

1. A ready to use teat dip composition comprising:
water;
from about 0.01-2% by weight iodine;
from about 0.01-3% by weight iodide ion; and
from about 35-75% by weight of an additive selected from the group consisting of glycerin and a mixture of at least about 10% by weight glycerin based upon the total weight of the composition and one or more C2-C7 polyhydric alcohols different than glycerin and each containing at least 2 hydroxyl groups, or polyethylene glycol, said composition containing no more than about 45% by weight propylene glycol when propylene glycol is one of said polyhydric alcohols.

2. The composition of claim 1, said iodine content being from about 0.2-1.1% by weight.

3. The composition of claim 1, said iodide ion content being from about 0.25-1.5% by weight.

4. The composition of claim 1, said additive being present from 40-65% by weight.

5. The composition of claim 1, said additive being glycerin.

6. The composition of claim 1, said additive comprising a mixture including at least about 10% by weight of glycerin based upon the total weight of the composition and a quantity of a C2-C7 polyhydric alcohol, or polyethylene glycol.

7. The composition of claim 1, said composition being stable during storage at ambient temperature over a period of at least about 3 months.

8. The composition of claim 7, said period being at least about 6 months.

9. The composition of claim 1, said composition having a free iodine content of from about 1-60 ppm at a temperature of 25° C.

10. The composition of claim 1, said composition having a free iodine concentration of from about 0.5-20 ppm at a temperature of 4° C.

11. The composition of claim 1, said composition having a freezing point of below about −15° C.

12. The composition of claim 11, said freezing point being below about 19° C.

13. The composition of claim 1, said composition further comprising at least one ingredient selected from the group consisting of thickening agents, surface active agents, buffering agents, and colorants.

14. A method of treating cow's teats comprising the step of applying the composition of claim 1 to said teats.

15. The method of claim 14, said applying step selected from the group consisting of dipping, spraying or foaming the composition of claim 1 onto said teats.

16. The method of claim 15, said applying step being carried out during periods where the ambient temperature is below about +5° C.

17. The method of claim 16, said temperature being below about 0° C.
